(19) United States
(12) Patent Application Publication
(10) Pub. No.: US 2007/0218537 A1
(43) Pub. Date: Sep. 20, 2007

(54) Method For Treating Ammonia-Containing Wastewater

(75) Inventors: Kenji Furukawa, Kumamoto (JP); Hiroyuki Tokito, Fukuoka-shi (JP)

Correspondence Address: THE WEBB LAW FIRM, P.C., 700 KOPPERS BUILDING, 436 SEVENTH AVENUE, PITTSBURGH, PA, 15219, US

(73) Assignee: Kumamoto Technology and Industry Foundation, Kamimashiki-gun (JP)

(21) Appl. No.: 10/594,800

(22) PCT Filed: Mar. 30, 2005
(86) PCT No.: PCT/JP05/06181

§ 371 Date: Sep. 28, 2006

(30) Foreign Application Priority Data
Mar. 30, 2004 (JP) 2004-100414

(51) Int. Cl. C12N 1/20

(52) U.S. Cl. 435/252.1

(57) ABSTRACT

A process for treating ammonia containing wastewater includes bringing an ammonia-treating material and ammonia containing wastewater into contact with each other to remove ammonia in the wastewater continuously as nitrogen gas, the ammonia-treating material including a long carrier and complex bacterial sludge attached and immobilized on the biomass carrier, the carrier including a net, a nonwoven fabric or a woven fabric of fibers or filaments, the carrier being attached to a support, the complex bacterial sludge containing bacterial sludge including autotrophic anammox bacteria and bacterial sludge including autotrophic ammonia-oxidizing bacteria, the ammonia containing wastewater containing dissolved oxygen at a concentration of not less than 0.5 mg/l. The process for treating ammonia containing wastewater uses the treating material in which the bacterial sludge are attached and immobilized, and nitritation and anammox reaction can take place efficiently and economically even when the wastewater contains dissolved oxygen at a high concentration.

### FIELD OF THE INVENTION

The present invention relates to a process for treating ammonia containing wastewater, more particularly to a process for treating ammonia containing wastewater using autotrophic ammonia-oxidizing bacteria and autotrophic denitrifying bacteria.

### BACKGROUND OF THE INVENTION

We have been forced to change our lifestyle from the mass production, mass consumption and mass disposal one in the 20th century to the recycling and low-load way of life. The spread of wastewater treatment services improves the quality of wastewater discharged to public water areas year by year. However, closed water bodies such as lakes and inland seas often have increased concentrations of nutrient salts such as nitrogen and phosphorous. The consequent eutrophication phenomena such as red tides are social problems. Accordingly, there is a need for an advanced, efficient and economic process capable of reducing organic matters and nutrient salts such as nitrogen and phosphorous in wastewater.

There are two general biological processes for removing nitrogen in wastewater: one is nitrogen removal by biological uptake and the other is nitrogen removal using nitrification and denitrification.

In the former process, bacteria assimilate nitrogen as they grow. However, treating wastewater over time results in accumulation of bacteria in an apparatus, causing the need of eliminating and disposing the bacteria. The bacteria disposed cause a waste problem.

The latter process oxidizes ammonia-nitrogen (NH₄—N) into nitrite-nitrogen (NO₂—N) with ammonia-oxidizing bacteria such as Nitrosomonas, and NO₂—N into nitrate-nitrogen (NO₃—N) with nitrite-oxidizing bacteria such as Nitrobacter under aerobic conditions, and reduces NO₃—N into nitrogen gas (N₂) with denitrifying bacteria under anaerobic conditions.

The typical nitrification-denitrification processes such as nitrification-denitrification with circulation of nitrified liquid, and A₂O process are only capable of approximately up to 80% total nitrogen removal. Although the triplex processes are expected to enable high total nitrogen removal, they use heterotrophic denitrifying bacteria and require external supply of carbon sources such as methanol, increasing costs. Accordingly, there is a need for the development of an economic nitrogen removal process as a replacement for the conventional nitrification-denitrification processes.

Graaf, et al. found Anammox bacteria, anaerobic autotrophic denitrifying bacteria capable of reducing NH₄—N and NO₂—N into N₂ gas. The bacteria are utilized in an ammonia-nitrogen removing reaction called Anammox reaction, which can remove nitrogen from wastewater at cheap treatment cost than the conventional nitrification-denitrification processes. While the conventional denitrifying bacteria are heterotrophic, the Anammox bacteria are autotrophic and do not require supply of external carbon sources, being economic.

To utilize the Anammox reaction in the removal of nitrogen in wastewater treatment, half of NH₄⁺ (in terms of mole) in the wastewater has to be oxidized into NO₂⁻ with aerobic autotrophic ammonia-oxidizing bacteria. This nitritation is represented by Formula (1):
NH₄⁺+1.5O₂→NO₂⁻+H₂O+2H⁺ (1)

After the nitritation, NH₄⁺ remaining in the wastewater and NO₂⁻ produced in the nitritation (Formula (1)) are converted by the Anammox reaction with autotrophic denitrifying bacteria under anaerobic conditions, as represented by Formula (2):
NH₄⁺+NO₂⁻→N₂+2H₂O (2)

As described above, the nitritation with autotrophic ammonia-oxidizing bacteria and the Anammox reaction with autotrophic denitrifying bacteria convert NH₄⁺ in the wastewater into N₂ gas.

At present, there are only a few Anammox reactions in practical use. This is because, for example, (1) the autotrophic anammox bacteria have an extremely slow growth rate, (2) NH₄—N and NO₂—N have to form an equimolar mixture for the Anammox reaction to proceed smoothly, but controlling these amounts is not easy, and (3) the reaction utilizes aerobic bacteria and anaerobic bacteria and therefore at least two reaction tanks are required for aerobic nitritation and anaerobic anammox reactions, making the apparatus large-scale. To address the problem (3), performing the nitritation and Anammox reactions in a single reaction tank in one step requires controlling aerobic and anaerobic conditions.

Patent Document 1 discloses a process in which approximately half of NH₄—N in a liquid phase is oxidized into NO₂—N, and NH₄—N and NO₂—N in the liquid phase are brought into contact with bacteria in the absence of oxygen and are converted into N₂ gas, which is removed from the system.

However, oxidizing approximately half of NH₄—N in the liquid phase and converting it perfectly into NO₂—N entail difficult control of conditions. Furthermore, the reaction requires two steps, which are a nitritation step and a anammox step.

Patent Document 2 discloses that a nitrogen removing reaction is performed in a single reaction tank. In a first denitrification step, slightly aerobic conditions are creased in the reaction tank and partial denitrification is performed in the presence of autotrophic nitrifying bacteria and autotrophic denitrifying bacteria. In a second denitrification step, denitrification is carried out in the presence of autotrophic denitrifying bacteria under anaerobic conditions.

The slightly aerobic conditions in the reaction tank in the first step probably hinder the function of the aerobic nitrifying bacteria. The slightly aerobic conditions can adversely affect the growth and activity of the anaerobic autotrophic anammox bacteria. Consequently, the treatment loading rate is reduced.

Patent Document 3 performs nitritation and Anammox reaction in a single reaction tank in one step with use of biological sludge that includes autotrophic anammox bacteria covered with autotrophic ammonia-oxidizing bacteria. The biological sludge including the bacteria is supported on particle-shaped sponge carriers, and the bacteria form distinctive phases that occur naturally. Specifically, the carrier surface is aerobic, and the autotrophic ammonia-oxidizing bacteria grow. The inside of the carriers is anaerobic, and the autotrophic denitrifying bacteria grow.

The patent document describes that in the nitrogen removing reaction, dissolved oxygen in the wastewater diffuses to the sponge carriers but is consumed by the nitritation by the autotrophic ammonia-oxidizing bacteria on the surface of the carriers. Consequently, the dissolved oxygen does not diffuse into the carriers and the inside of the carriers maintains anaerobic conditions, and the Anammox reaction by the autotrophic denitrifying bacteria takes place.

However, because the particle-shaped sponge supporting the biological sludge is moved with the flow of the wastewater, the oxygen concentration in the wastewater does not change during the treatment. When excessive oxygen is supplied, the excess oxygen that is not consumed in the nitritation by the autotrophic ammonia-oxidizing bacteria diffuses into the carriers and inhibits the growth of the anaerobic autotrophic anammox bacteria. Accordingly, the nitrogen removing reaction using the particle-shaped sponge carriers has a problem that the supply of oxygen-containing gas is limited.

Patent Document 4 discloses a process for treating ammonia containing wastewater. The process includes a first step in which NH₄—N is oxidized with ammonia-oxidizing bacteria at a pH of not more than 7.2 by controlling the aeration, and a second step in which NH₄—N and the oxidation product are converted into N₂ with denitrifying bacteria. The patent document describes that the first step and the second step are carried out simultaneously in a single bioreactor, wherein ammonia-oxidizing bacteria and denitrifying bacteria are present in a solid phase, the ammonia-oxidizing bacteria are substantially present in the outer aerobic part of the solid phase, and the denitrifying bacteria are substantially present in the inner anaerobic part of the solid phase.

In the first and the second step in the single bioreactor, oxygen is supplied in a limited amount. Consequently, as with the treatment process of Patent Document 2, the reaction by the aerobic ammonia-oxidizing bacteria will not proceed smoothly. Slight amounts of dissolved oxygen can adversely affect the growth and activity of the anaerobic anammox bacteria. Consequently, the wastewater treatment load is reduced. The patent document describes that the solid phase may be a biofilm-carrying particulate carrier or a biofilm-carrying immobilizing carrier, but does not disclose specific examples thereof.

As described hereinabove, there is a need for a process for treating ammonia containing wastewater whereby the nitritation and the Anammox reaction are performed efficiently and economically, using a treating material in which autotrophic ammonia-oxidizing bacteria and autotrophic denitrifying bacteria are attached and immobilized, without limiting the supply of oxygen even if the wastewater has a high concentration of dissolved oxygen.

Patent Document 1: JP-A-2001-37467

Patent Document 2: JP-A-2003-126888

Patent Document 3: JP-A-2001-293494

Patent Document 4: JP-A-2001-506535

### DISCLOSURE OF THE INVENTION

### Problems to be Solved by the Invention

The present invention is directed to solving the above conventional problems. It is therefore an object of the invention to provide a process for treating ammonia containing wastewater by bringing ammonia containing wastewater into contact with a specific ammonia-treating material to remove ammonia in the wastewater continuously as nitrogen gas.

### MEANS FOR SOLVING THE PROBLEMS

The present inventors diligently studied to solve the above problems and found that ammonia containing wastewater which contains dissolved oxygen at a high concentration is efficiently treated by bringing it into contact with a specific ammonia-treating material.

A process for treating ammonia containing wastewater according to the present invention comprises bringing an ammonia-treating material and ammonia containing wastewater into contact with each other to remove ammonia in the wastewater continuously as nitrogen gas,

the ammonia-treating material comprising a long carrier and complex bacterial sludge attached and immobilized on the carrier, the carrier comprising a net, a nonwoven fabric or a woven fabric comprising fibers or filaments, the carrier being attached to a support, the complex bacterial sludge comprising bacteria including autotrophic anammox bacteria and bacteria including autotrophic ammonia-oxidizing bacteria,

the ammonia containing wastewater containing dissolved oxygen at a concentration of not less than 0.5 mg/l.

Preferably, the bacteria including autotrophic anammox bacteria are attached and immobilized on the fibers or filaments, and the bacteria including autotrophic ammonia-oxidizing bacteria are attached and immobilized on an outer surface of the bacteria including autotrophic anammox bacteria.

Preferably, in the complex bacterial sludge, the bacteria including autotrophic anammox bacteria are present within the bacteria including autotrophic ammonia-oxidizing bacteria.

Preferably, the ammonia-treating material and the ammonia containing wastewater are brought into contact with each other in one step.

Preferably, the ammonia-treating material and the ammonia containing wastewater are brought into contact with each other while supplying air to the ammonia containing wastewater.

Preferably, the ammonia-treating material is provided in an inner peripheral area in a reaction tank, the ammonia containing wastewater is supplied to the reaction tank, and air is supplied from a central bottom part of the reaction tank to achieve a dissolved oxygen concentration of not less than 0.5 mg/l.

Preferably, air is supplied from a central bottom part of the reaction tank to generate an upward flow of wastewater in a central area of the reaction tank and a downward flow of wastewater in an inner peripheral area of the reaction tank.

Preferably, an air guide tube is provided in a central area in the reaction tank in a position such that a lower opening of the tube is opposed to the bottom of the reaction tank with a space from the bottom of the reaction tank, and air is supplied through the lower opening of the air guide tube to generate an upward flow of wastewater in the central area in the reaction tank.

Preferably, the longer direction of the long carrier is perpendicular to the bottom of the reaction tank.

Preferably, the fibers or the filaments are polyacrylic fibers or polyacrylic filaments.

Preferably, the long carrier has a length to diameter ratio of not less than 3.

Preferably, the bacteria including autotrophic ammonia-oxidizing bacteria are attached and immobilized in a thickness of not less than 5 mm.

Preferably, the ammonia containing wastewater in the reaction tank has a BOD concentration of not more than 20 mg/l, a temperature of 30 to 40° C., or a pH of 7.4 to 8.0.

### EFFECTS OF THE INVENTION

The treating material includes the autotrophic ammonia-oxidizing bacteria and autotrophic anammox bacteria attached and immobilized on the specific long carrier. The process for treating ammonia containing wastewater according to the invention uses the treating material, and nitritation and Anammox reaction can take place efficiently and economically even when the wastewater contains high dissolved oxygen concentration.

### BRIEF DESCRIPTION OF THE DRAWINGS

FIG. 1 is a picture showing a polyacrylic net used in Examples of the invention;

FIG. 2 is a schematic view showing a reaction apparatus used in Examples of the invention;

FIG. 3 is a picture showing an ammonia-treating material in a reaction tank used in Examples of the invention;

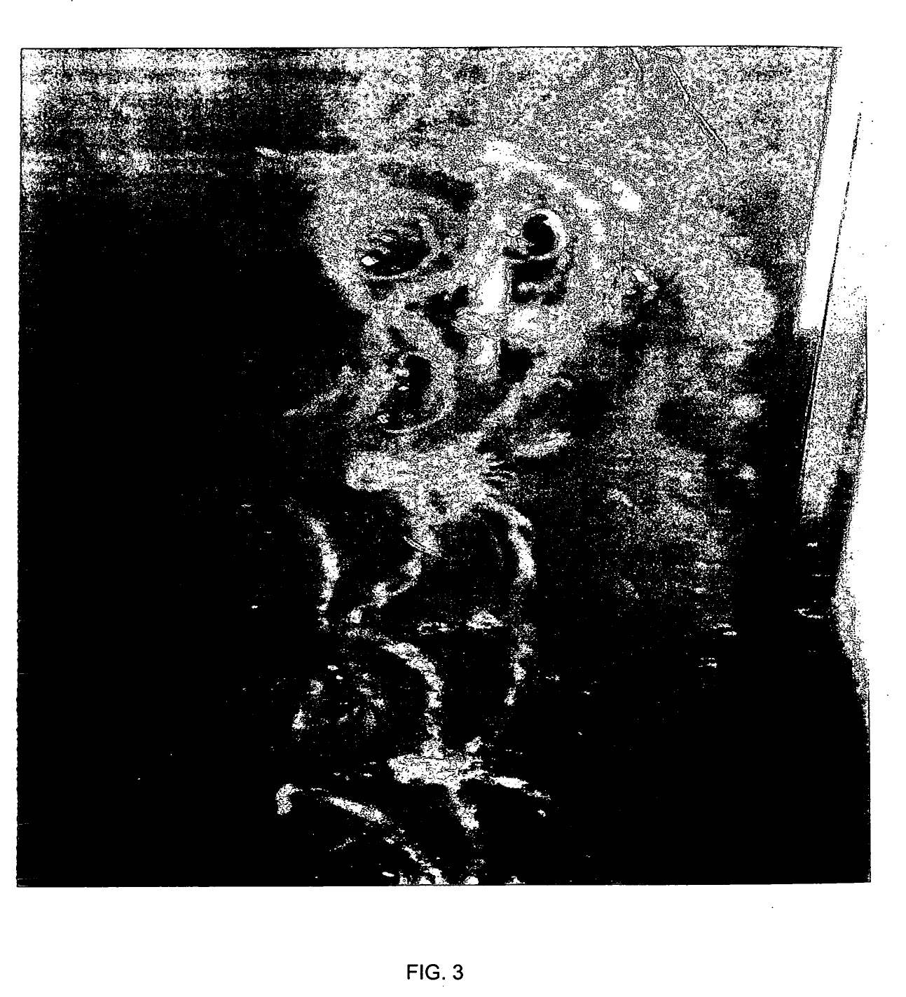

FIG. 4 is a graph of concentrations of nitrogen in various forms in wastewater effluent during continuous treatment in Examples of the invention;

FIG. 5 is a graph showing nitrogen removal in wastewater effluent during continuous treatment in Examples of the invention;

FIG. 6 is a graph of concentrations of nitrogen in various forms in wastewater effluent in Examples of the invention;

FIG. 7 is a graph showing nitrogen removal in wastewater effluent in Examples of the invention;

FIG. 8 is a graph showing NH₄—N removal in wastewater effluent in Examples of the invention;

FIG. 9 is a graph showing concentrations of dissolved oxygen (DO) in wastewater in the reaction tank in Examples of the invention;

FIG. 10 is a graph showing pH of wastewater in the reaction tank in Examples of the invention;

FIG. 11 is a photomicrograph by the FISH method of bacteria that grew in the reaction tank in Examples of the invention;

FIG. 12 is a confocal laser scanning microphotograph of bacteria that grew in the reaction tank in Examples of the invention;

FIG. 13 is a confocal laser scanning microphotograph of bacteria that grew in the reaction tank in Examples of the invention;

FIG. 14 is a graph of NO₃—N concentrations and nitrogen removal in wastewater effluent in Examples of the invention;

FIG. 15 is a graph of concentrations of nitrogen in various forms in wastewater effluent in Examples of the invention;

FIG. 16 is a graph showing NH₄—N loading and nitrogen removal rates in wastewater effluent in Examples of the invention;

FIG. 17 is a graph of concentrations of nitrogen in various forms in wastewater effluent in Examples of the invention; and

FIG. 18 is a graph showing nitrogen removal in wastewater effluent in Examples of the invention.

### DESCRIPTION OF CODES

1: reaction tank
2: ammonia-treating material
3: wastewater
4: wastewater inlet
5: air inlet
6: air guide tube
7: effluent outlet
8: pH controller
9: temperature controller
10: upper air phase
11: support
12: wastewater flow

### PREFERRED EMBODIMENTS OF THE INVENTION

The process for treating ammonia containing wastewater according to the present invention will be described in detail hereinbelow.

1. Ammonia-Treating Material

The ammonia-treating material in the invention includes a long biomass carrier composed of a net, a nonwoven fabric or a woven fabric of fibers or filaments. Bacteria are attached and immobilized on the carrier.

[Long Carrier]

The long carrier is composed of a net type fabrics, a nonwoven fabric or a woven fabric.

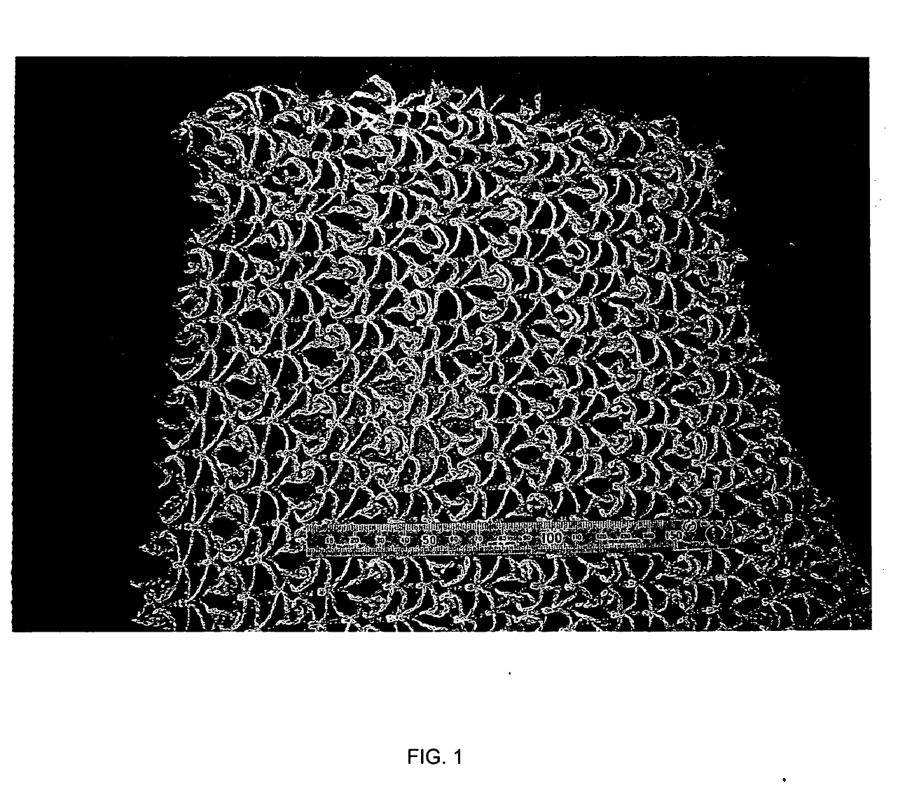

FIG. 1 shows an example of the net. The net shown is a specially-knitted three-dimensional structure, and its skeleton is made of filaments. The skeleton includes strands of a high-absorption bulky polymer that are knitted in the skeleton and are uniformly dispersed. The net has a high porosity and is bulky, and therefore laminating the nets gives a long carrier with a desired volume. The knitted net has high contraction and expansion properties. Consequently, the net may be contracted and fitted in a support such as a frame, and the density of the carrier may be easily controlled.

Examples of the fibers and filaments for making the nets include fibers and filaments of metals, polymers, coconut and palm. Polymer filaments are preferable for their high contraction and expansion properties, excellent durability, lightweight and inexpensiveness. The polymer filaments include polyethylene filaments, polypropylene filaments, polyester filaments, polyurethane filaments, polyamide filaments and polyacrylic filaments. Of these, polyacrylic filaments are most preferable because they have highest affinity for water and permit good attachment and immobilization of the bacteria.

Specifically, a net of polyacrylic filaments (trade name: Biofix, manufactured by NET) is preferable.

The nonwoven fabric may be fabricated by blowing a molten polymer through a small-diameter nozzle to disperse fibers or filaments, followed by fixing the fibers or filaments. Preferably, the fibers or filaments are dispersed and fixed to form a sheet having a uniform density.

The materials of the fibers and filaments for making the nonwoven fabrics include polyethylenes, polypropylenes, polyesters, polyurethanes, polyamides and polyacryl. They have excellent mechanical strength, chemical resistance and durability, and are lightweight and inexpensive. Of the above materials, polyesters and polypropylenes are more preferable for their superior forming properties and strength, and small fiber diameters. Polyester nonwoven fabrics (for example, products of Japan Vilene Company, Ltd.) are most preferable for an additional capability of permitting good attachment and immobilization of the bacteria.

Preferably, the nonwoven fabric is not less than 5 mm in thickness, and several nonwoven fabrics form a bulky structure in which they are crossed in the middle and are joined together to have a chrysanthemum cross section.

The woven fabrics may be made by weaving the fibers or filaments.

The materials of the fibers and filaments for making the woven fabrics include polyethylenes, polypropylenes, polyesters, polyurethanes, polyamides and polyacryl.

The long carrier which is the above-described net, nonwoven fabric or woven fabric has an appropriate porosity, whereby the carrier permits good attachment and immobilization of the bacteria to increase the wastewater treatment efficiency. The balance is good between the amount of the wastewater diffused into the bacteria and the amount of the bacteria on the carrier. Consequently, an aerobic area and an anaerobic area are maintained favorably.

The long carrier composed of the net, nonwoven fabric or woven fabric is attached to a support in the reaction tank. Examples of the supports include bars, frames, rigid meshes, porous materials, partition boards and tubular materials.

The long carrier is preferably fitted and fixed in a hollow frame having excellent shape stability and high rigidity, whereby the shape of the net, nonwoven fabric or woven fabric is stabilized and the long carrier is easily installed in and removed from the reaction tank.

The materials of the supports include metals and polymers, with the polymers being preferable for their non-corrosion properties. Examples of the polymers as supports include polyethylenes, polypropylenes, polyvinyl chlorides, unsaturated polyesters, polyamides and ABS resins.

The diameter and length of the long carrier are not particularly limited. The length to diameter ratio is desirably not less than 3, preferably not less than 5, more preferably 10, in which case the ammonia-treating material and the wastewater having a high dissolved-oxygen concentration are favorably contacted with each other. The diameter of the long carrier refers to a diameter when the long carrier is a cylindrical column, and a minor axis when it is rectangular. When the diameter is excessively small, the autotrophic anammox bacteria may be exposed to aerobic conditions and the activity of the autotrophic anammox bacteria may be hindered.

[Ammonia-Treating Material]

The ammonia-treating material includes the above-described long carrier, and complex bacterial sludge attached and immobilized on the carrier. The complex bacterial sludge include bacteria including autotrophic anammox bacteria (hereinafter, simply autotrophic anammox bacteria) and bacteria including autotrophic ammonia-oxidizing bacteria (hereinafter, simply autotrophic ammonia-oxidizing bacteria).

More specifically, it is preferable that the autotrophic anammox bacteria be attached and immobilized on the fibers or filaments, and the autotrophic ammonia-oxidizing bacteria be attached and immobilized on the outer surface of the autotrophic anammox bacteria. Also preferably, in the complex bacterial sludge, the autotrophic anammox bacteria are present within the autotrophic ammonia-oxidizing bacteria, and the complex bacterial sludge are attached and immobilized on the fibers or filaments. In the complex bacterial sludge, the autotrophic anammox bacteria may form dispersed phases. In particular, the autotrophic anammox bacteria preferably form a core and the autotrophic ammonia-oxidizing bacteria preferably form a sheath, that is, the complex bacterial sludge preferably have a core-sheath structure.

The ammonia-treating material may include other bacteria such as nitrifying bacteria, heterotrophic bacteria and non-organisms in addition to the autotrophic ammonia-oxidizing bacteria and the autotrophic anammox bacteria. Each of the bacteria may comprise a single bacterium or may include two or more kinds of bacteria, other organisms and non-organisms.

The configuration of the autotrophic ammonia-oxidizing bacteria and autotrophic anammox bacteria that are attached and immobilized is not particularly limited. Examples of the configurations include rectangles, cylindrical columns, polygonal columns, configurations including part of these shapes, and amorphous shapes. Of these, cylindrical columns and polygonal columns such as hexagonal columns are preferred.

The autotrophic ammonia-oxidizing bacteria that are attached and immobilized preferably have a thickness of not less than 5 mm, preferably not less than 10 mm, more preferably not less than 20 mm. The autotrophic ammonia-oxidizing bacteria having this thickness maintain anaerobic conditions for the autotrophic anammox bacteria.

The total thickness of the autotrophic ammonia-oxidizing bacteria and autotrophic anammox bacteria is preferably not less than 10 mm.

The bacteria are formed as a result of growth of the bacteria, and therefore the density thereof on the carrier is usually uncontrollable. When the bacteria density is increased, the frequent result is that the balance of the aerobic area and the anaerobic area is lowered, resulting in lowered treatment efficiency. In the invention, the long carrier which is the net, nonwoven fabric or woven fabric permits an appropriate bacteria density and prevents the decrease in treatment efficiency.

A preferred production of the ammonia-treating material will be described below. Sludge including the autotrophic ammonia-oxidizing bacteria and autotrophic anammox bacteria is attached and immobilized on the long carrier fitted to the support. Ammonia containing wastewater is supplied, and nitritation is continuously performed by the autotrophic ammonia-oxidizing bacteria. As a consequence, the autotrophic anammox bacteria are within the autotrophic ammonia-oxidizing bacteria.

The following process is also preferable. Sludge including the autotrophic anammox bacteria is dispersed in water or wastewater having a dissolved-oxygen concentration of 0 mg/l or nearly 0 mg/l. The dispersion of the sludge in water or wastewater is supplied to a reaction tank in which the long carrier attached to the support is provided, and the autotrophic anammox bacteria are attached and immobilized. In the supply of water or wastewater, the water or wastewater is circulated by feeding an oxygen-free gas such as nitrogen or by stirring with a stirrer. Subsequently, water or wastewater in which sludge including the autotrophic ammonia-oxidizing bacteria is dispersed is supplied while being circulated in a manner as described above. Consequently, the autotrophic ammonia-oxidizing bacteria are attached and immobilized on the outer surface of the autotrophic anammox bacteria.

Addition of inorganic salts to the ammonia containing wastewater or the water or wastewater for the attached—immobilization of the autotrophic anammox bacteria increases the growth rate of the autotrophic anammox bacteria. Examples of the inorganic salts include potassium chloride, sodium chloride, calcium chloride, magnesium chloride, zinc chloride, ferrous chloride, ferric chloride, potassium sulfate, sodium sulfate, calcium sulfate, magnesium sulfate, iron sulfate, EDTA and mixtures thereof. Seawater is an inexpensive source of inorganic salts.

The amount of the inorganic salts is preferably in the range of 0.1 to 5 g/l in order to remarkably increase the growth rate of the autotrophic anammox bacteria.

[Ammonia Containing Wastewater]

In the process for treating ammonia containing wastewater according to the present invention, ammonia containing wastewater is brought into contact with the ammonia-treating material.

The ammonia containing wastewater used in the invention is not particularly limited as long as it is industrial or domestic wastewater rich in NH₄—N. Examples thereof include digestive liquors, sludge dehydration filtrates, secondary effluents of night soil treatment, livestock wastewater, effluents from methane fermentation of livestock wastewater, waste effluents, and denitrification effluents in factories and power plants. Preferably, wastewater contains much NH₄—N and is subjected to a primary treatment such as activated sludge treatment to reduce organic matters inasmuch as the biological oxygen demand (BOD) is not more than 300 mg/l and the C/N ratio is low. The BOD in the ammonia containing wastewater is more preferably not more than 20 mg/l, optimally not more than 10 mg/l.

2. Process for Treating Wastewater

[Process for Treating Ammonia Containing Wastewater]

In the process for treating ammonia containing wastewater of the invention, the ammonia containing wastewater having a high dissolved-oxygen concentration is brought into contact with the ammonia-treating material, whereby NH₄—N in the wastewater is continuously removed in an end form of N₂ gas.

The contact preferably takes place in a reaction tank. The invention may preferably use a conventional reaction tank that is a circular cylinder long in the height direction. Alternatively, reaction tanks may be polygonal in cross section such as triangular, rectangular, pentagonal or hexagonal. A hexagonal cross section is most preferable because it is nearly circular and permits high efficiency of wastewater treatment. To achieve higher efficiency of wastewater treatment, a plurality of partition walls in a honeycomb form may be provided in the reaction tank.

The treatment using the reaction tank may be performed in one step in which the wastewater is treated in a single reaction tank, or may be performed in multi steps in which the wastewater is treated through a plurality of reaction tanks. Treatment in multi steps is capable of high treatment rate and high total nitrogen removal. Preferably, the nitritation with the autotrophic ammonia-oxidizing bacteria and the anammox reaction with the autotrophic anammox bacteria take place in a single reaction tank. A plurality of reaction tanks may be arranged in a line to enable treatment of high volume of wastewater and continuous treatment without interruption during repair and check of the reaction tank.

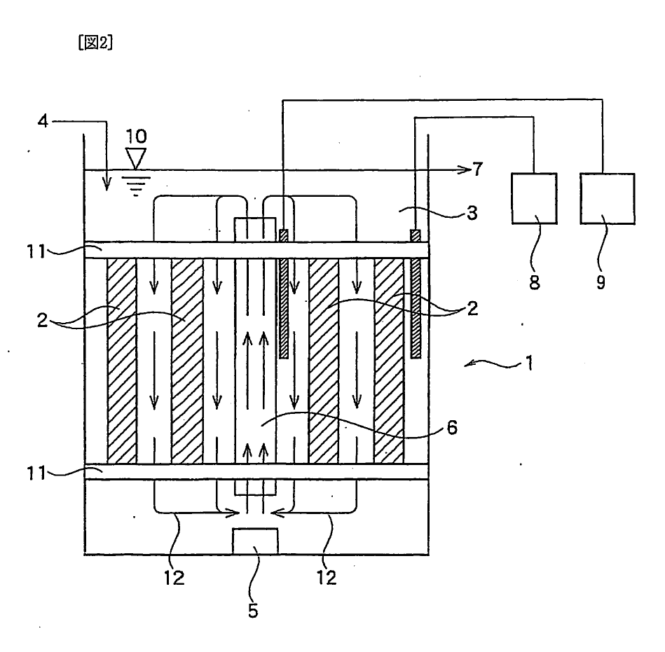

The process for treating ammonia containing wastewater according to the present invention will be described in detail with reference to a schematic view of a reaction apparatus shown in FIG. 2. A reaction tank 1 includes ammonia-treating materials 2 attached to supports 11. There may be one or more ammonia-treating materials 2. The ammonia-treating materials 2 are preferably provided in an inner peripheral area in the reaction tank. The inner peripheral area in the reaction tank is within a range of up to 70% inward, preferably up to 90% inward from the outer periphery of the reaction tank 1, relative to the distance from the outer wall to the center of the reaction tank 1.

Ammonia containing wastewater 3 is supplied from a wastewater inlet 4. Treated wastewater 3 is discharged from the effluent outlet 7. To make it sure that the wastewater 3 is treated, that is, to prevent short circuit of the wastewater, a partition wall (not shown) is preferably provided between the wastewater inlet 4 and the effluent outlet 7 to permit communication only at a bottom part in the reaction tank 1.

The wastewater 3 may be supplied continuously. The supply may be determined appropriately depending on wastewater treatment conditions. To obtain high total nitrogen removal, the supply is preferably in the range of 0.1 to 1 kg NH₄—N/m³/day. Herein, kg is the total amount of NH₄—N in the wastewater supplied, and m³ is the volume of the reaction tank.

The wastewater 3 supplied in the reaction tank 1 is brought into contact with the ammonia-treating materials 2 in which the bacteria are attached and immobilized on the long carriers. Consequently, nitrogen removal reaction takes place. Specifically, NH₄—N in the wastewater 3 is oxidized into NO₂—N by the autotrophic ammonia-oxidizing bacteria that are attached and immobilized on the carriers. Subsequently, NH₄—N remaining in the wastewater 3 and NO₂—N are converted into N₂ gas by autotrophic anammox bacteria that are attached and immobilized on the carriers. In this manner, NH₄—N in the wastewater 3 is continuously removed as N₂ gas.

The wastewater treatment in the invention is performed under aerobic conditions, namely in the presence of oxygen dissolved in the wastewater 3 in the reaction tank 1, and preferably while supplying air in the wastewater 3 in the reaction tank 1. Air may be replaced by oxygen or an oxygen-containing gas, but air itself is preferable. As used herein, air comprehends oxygen and oxygen-containing gas.

Air is preferably supplied in the wastewater 3 through a central bottom area of the reaction tank 1. Accordingly, an air inlet 5 is preferably provided at a central bottom area of the reaction tank 1. The central bottom area is in a range of 30% outward, preferably 10% outward from the center of the reaction tank, relative to the distance from the outer wall to the center of the reaction tank.

Oxygen in air dissolves in the wastewater 3 as bubbles rise in the reaction tank 1. The dissolution rate of oxygen in the wastewater 3 is low, and the dissolved oxygen content is preferably increased by increasing the height of the reaction tank 1, supplying microbubbles with small diameters, or using an auxiliary tank provided with a microbubble generator.

By supplying air, the dissolved oxygen concentration in the wastewater 3 is desirably not less than 0.5 mg/l, preferably not less than 1.5 mg/l, optimally not less than 2.0 mg/l. This dissolved oxygen concentration permits rapid nitritation by the aerobic autotrophic ammonia-oxidizing bacteria. When the dissolved oxygen concentration is extremely low, the attached and immobilized autotrophic ammonia-oxidizing bacteria are inhibited, and its biofilm thickness is reduced.

The wastewater 3 in the reaction tank 1 is preferably circulated such that an upward wastewater flow 12 is created in a central area in the reaction tank 1, and a downward wastewater flow 12 is produced in an inner peripheral area in the reaction tank 1. To crease these wastewater flows, an air guide tube 6 is preferably provided in a central area, and air is forcibly blown upward and aerates the wastewater to produce the wastewater flows 12. The lower opening of the air guide tube 6 is preferably away from the central bottom of the reaction tank 1 inasmuch as the upward wastewater flow 12 is formed. For example, it may be away from the central bottom by 10% the height of the reaction tank 1, whereby the wastewater flows 12 are favorably produced in the reaction tank 1 and the wastewater 3 may be circulated in the reaction tank 1 without a stirrer. The wastewater flows 12 produced by supplying air are gentle compared to the forced flows by stirring, and it is unlikely that the autotrophic ammonia-oxidizing bacteria and autotrophic anammox bacteria are detached from the long carriers during the wastewater treatment.

The longer direction of the long carriers is preferably perpendicular to the bottom of the reaction tank 1. As a result of the long carriers being provided as such, the contact of the wastewater 3 with the ammonia-treating materials 2 takes place favorably. Consequently, the wastewater treatment while aerating the wastewater 3 in the reaction tank 1 or while producing the wastewater flows 12 achieves high total nitrogen removal.

The treatment process according to the invention uses the ammonia-treating materials and produces the wastewater flows in the wastewater having a high dissolved oxygen concentration, and consequently achieves higher total nitrogen removal. The reason for this will be probably as follows. The bacteria are strongly attached and immobilized on the filaments forming long carrier nets that are attached to the supports, and are not detached from the carriers by the wastewater flows. Consequently, the nitritation and anammox reaction take place efficiently.

The nets or the like have an appropriate porosity, so that after the bacteria are attached and immobilized thereon, they may be attached to the supports in an appropriate density. Consequently, the wastewater penetrates the attached and immobilized bacteria. The circulation of the wastewater by aerating without forced stirring helps the wastewater penetrate the attached and immobilized bacteria. The oxygen transfer rate in the wastewater is low, and the dissolved oxygen in the wastewater is consumed by nitritation by the attached and immobilized autotrophic ammonia-oxidizing bacteria. Consequently, even in the event that the wastewater contains a high concentration of dissolved oxygen, anaerobic conditions are maintained for the autotrophic anammox bacteria. Meanwhile, NH₄—N, and NO₂—N produced by the nitritation are easily diffused into the attached and immobilized bacteria, and the anammox reaction by the autotrophic anammox bacteria takes place smoothly. As described above, the treatment process of the invention achieves high total nitrogen removal.

The treatment process of the invention uses the autotrophic ammonia-oxidizing bacteria and the autotrophic anammox bacteria in combination. Accordingly, controlling the wastewater temperature in the reaction tank 1, that is, the temperature of the bacterial reaction, is preferable for accelerating the reaction. The reaction temperature is usually in the range of 15 to 50° C., preferably 25 to 45° C., more preferably 30 to 40° C., optimally 32 to 38° C. This wastewater temperature activates the autotrophic ammonia-oxidizing bacteria and the autotrophic anammox bacteria, and the reaction is accelerated.

The reaction tank 1 is preferably provided with an automatic temperature controller 9 for keeping the wastewater temperature in the reaction tank 1 constant.

In the treatment process, the pH of the wastewater 3 is desirably adjusted to 7.0 to 9.0, preferably 7.4 to 8.0. When the pH is in this range, the autotrophic ammonia-oxidizing bacteria and the autotrophic anammox bacteria are very active, and the reaction is accelerated.

The pH of the wastewater 3 may be adjusted using inorganic compounds. Examples of the inorganic compounds include ammonium chloride, ammonium phosphate, potassium nitrite, potassium carbonate, potassium hydrogencarbonate, sodium nitrite, sodium carbonate and sodium hydrogencarbonate. Of these, sodium hydrogencarbonate is most preferable. The inorganic compounds are preferably supplied to the reaction tank 1 in the form of aqueous solutions.

It is preferable that the reaction tank 1 be designed such that the pH of the wastewater 3 in the reaction tank 1 is measured and automatically or manually adjusted to a desired level. To enable this pH adjustment, the reaction tank 1 is preferably provided with a pH controller 8.

The progress of the reaction in the reaction tank 1 is generally controlled by manipulating conditions such as supply of the wastewater in the reaction tank 1, wastewater temperature in the reaction tank 1, and pH of the wastewater 3 in the reaction tank 1. Accordingly, the wastewater 3 to be supplied in the reaction tank 1 is preferably analyzed beforehand to obtain data, particularly NH₄—N concentration, and the conditions are manipulated depending on the data. The reaction tank 1 is preferably provided with a controller (not shown) capable of automatic manipulation of the conditions to keep the nitrogen concentration in the treated wastewater 3 below a predetermined level.

The mean residence time of the wastewater 3 in the reaction tank is variable depending on the configuration of the reaction tank 1 and the supply of the wastewater. Generally, it is from 30 minutes to 30 hours, preferably from 1 to 20 hours, particularly preferably from 3 to 10 hours. When the mean residence time is in the above range, most NH₄—N in the wastewater 3 is converted to N₂ gas and is removed from the system.

The treatment process of the invention can remove approximately 90% of NH₄—N as N₂ gas, while about 5 to 10% of nitrogen components contained in the wastewater remains as NO₃—N. In the process, the bacteria are not drastically increased as in the activated sludge method, and the process does not require frequent withdrawal of excess sludge. Consequently, the process enables continuous treatment and is economic.

The present invention will be described in greater detail by examples below, but it should be construed that the invention is in no way limited thereto.

### EXAMPLES

Measurements in Examples were carried out by methods shown in Table 1.

TABLE 1

| Item | Method | Remarks |
|---|---|---|
| pH | Portable pH meter | pH in reactor was measured with |
| | (HACHEC 20 pH/ISE | NISSIN pH CONTROLLER |
| | Meter) | NPH-690D |
| ORP | Platinum electrode | UK-2030 manufactured by |
| | method | CENTRAL KAGAKU Corp |
| | | Portable ORP meter |
| NH₄—N | OPP method | V-1100 Hitachi ratio beam |
| | Indophenol method | spectrophotometer |
| | ( JIS K0102) | |
| NO₂—N | Ion chromatography | ION ANALYZER LA-100 |
| | (TOA ION ANALYZER | manufactured by Toa Denpa |
| | IA-100) | Kogyo Co., Ltd. |
| | Colorimetric | Spectrophotometer used for |
| | Method | NH₄—N |
| NO₃—N | Ion chromatography | ION ANALYZER LA-100 |
| | Ultraviolet | manufactured by Toa Denpa |
| | spectrophotometric | Kogyo Co., Ltd. |
| | screening | Spectrophotometer used for |
| | | NH₄—N |
| Alkalinity | Total alkalinity | Sewage testing method |
| DO conc. | Membrane electrode | HORIBA OM-51DO |
| | method | |

ORP: oxidation reduction potential
NH₄—N: ammonia-nitrogen
NO₂—N: nitrite-nitrogen
NO₃—N: nitrate-nitrogen
DO: dissolved oxygen

### Reference Example 1

### Production Example 1 of Ammonia-Treating Material

(Long Carrier)

A long carrier net composed of polyacrylic filaments as shown in FIG. 1 was used (trade name: Biofix, manufactured by NET). The net had properties shown in Table 2.

The long net was 100 mm in diameter and 330 mm in height, and was attached to a support 110 mm in length, 110 mm in width, and 330 mm in height.

TABLE 2
Acrylic bulky filaments

| Yarn | 2/10 | |
|---|---|---|
| Length | 23324 | m/m³ |
| Diameter | 2 | mm |
| Surface area | 146.5 | m²/m³ |

(Reaction Apparatus)

A reaction apparatus as illustrated in FIG. 2 was used. The apparatus included a tank that was made of an acrylic resin and was 450 mm in height, 150 mm in width, 115 mm in depth and 5.43 l in reaction part volume. Eight long carriers were attached to the supports, and were arranged in an inner peripheral area in the reaction tank. The longer direction of the carriers was perpendicular to the bottom of the reaction tank.

(Attachment and Immobilization of Autotrophic Ammonia-Oxidizing Bacteria and Autotrophic Anammox Bacteria)

The present inventors had a well acclimatized nitrifying activated sludge, which was cultivated by fill and draw method with synthetic sewage in a laboratory. 15 g of this nitrifying activated sludge was added to 5 L of water, and a mixed liquor suspended solid (MLSS) having a concentration of approximately 3000 mg/l was obtained. Table 3 shows the composition of influent water medium used in the acclimatization of nitrifying activated sludge and continuous nitritation test.

TABLE 3

| Component | Concentration | |
|---|---|---|
| (NH₄)₂SO₄ | 10 to 100 | mg-N/l |
| KH₂PO₄ | 13.6 | mg/l |
| C₆H₁₂O₆ | 20 | mg-C/l |

The aqueous solution of the nitrifying activated sludge (MLSS concentration: approximately 3000 mg/l) was supplied to the reaction tank. Air was continuously supplied at 1.7 mg-O₂/l from a central bottom part of the reaction tank. The pH in the reaction tank was controlled with a pH controller (NPH-690D), and the water temperature in the reaction tank with a thermostat. The pH was adjusted by automatic addition of a 0.5 mol/l NaHCO₃ solution. After the nitrifying activated sludge was added, the mixed liquor was circulated by aeration. The nitrifying activated sludge was substantially attached and immobilized on the long carriers in about 4 hours. The results are shown in Table 3.

The nitrifying activated sludge that was attached and immobilized was acclimatized for 100 days, while the influent NH₄—N concentration in the influent water medium was gradually increased from 20 mg/l to 100 mg/l, and the mean residence time was gradually decreased from 12 hours to 6 hours. Consequently, an ammonia-treating material (A) was produced.

Continuous nitritation test was performed using the ammonia-treating material (A). Optimum conditions were found to be a pH of 7.5, a water temperature in reaction tank of 35° C., and a mean residence time of 6 hours.

### Example 1

Ammonia containing wastewater having a NH₄—N concentration of 100 mg/l was continuously treated for 40 days using the ammonia-treating material (A) produced in Reference Example 1, at a pH of 7.5 and a water temperature in reaction tank of 35° C., and with a mean residence time of 5 hours. An inorganic salt medium shown in Table 4 was added on the 25th day from the initiation of the continuous treatment.

TABLE 4

| Component | Concentration | |
|---|---|---|
| KCl | 1400 | mg/l |
| NaCl | 1000 | mg/l |
| CaCl₂ | 1900 | mg/l |
| MgSO₄•7H₂O | 2000 | g/l |

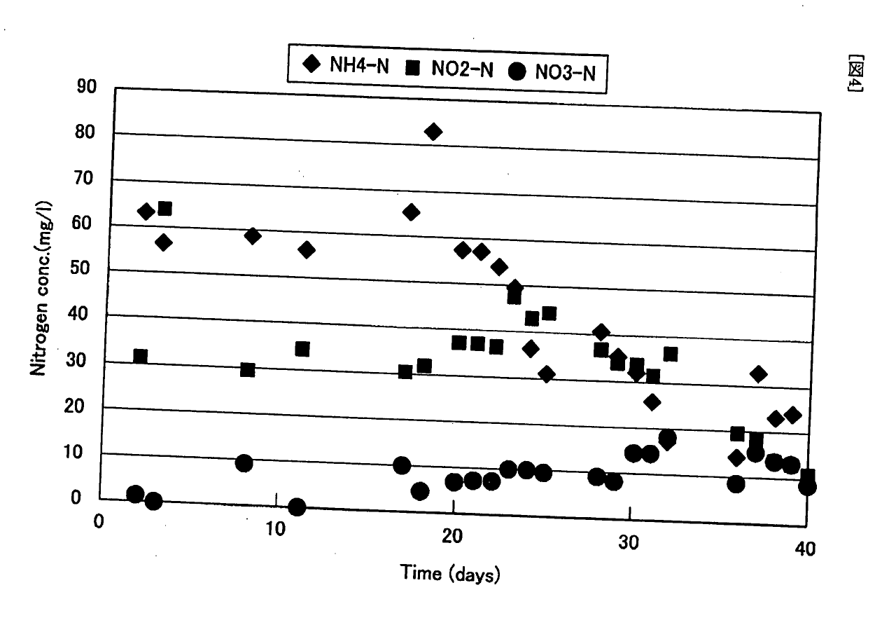

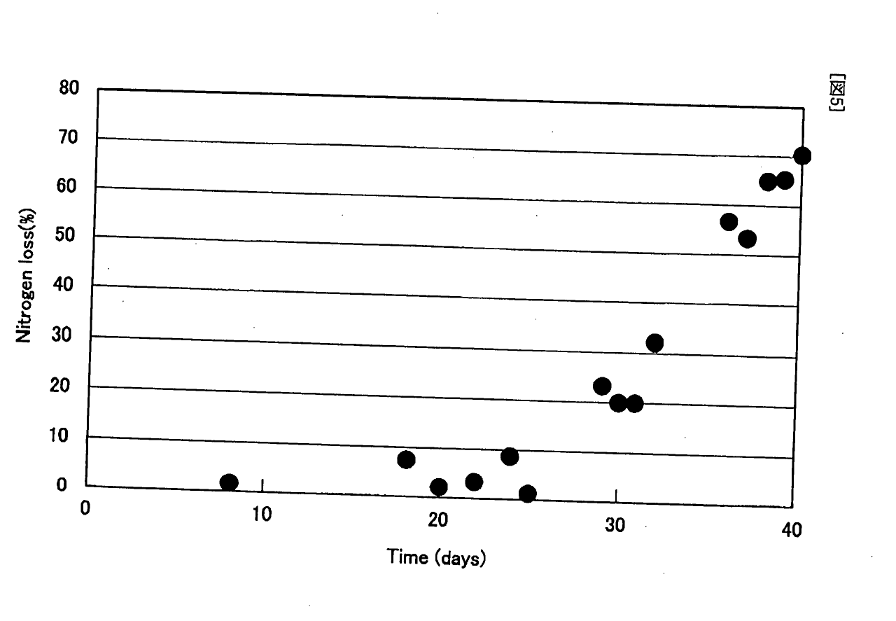

FIG. 4 shows concentrations of NH₄—N, NO₂—N and NO₃—N in wastewater effluent during the continuous treatment, and FIG. 5 shows nitrogen removal (%). After the addition of inorganic salt medium on the 25th day, the NH₄—N and NO₂—N concentrations reduced, and the nitrogen removal increased, indicating the progress of the anammox reaction. This result showed that the autotrophic ammonia-oxidizing bacteria and the autotrophic anammox bacteria had been attached and immobilized.

The wastewater was continuously treated for another 110 days, that is, a total of 150 days combined with the first 40 days. The wastewater treatment conditions were as follows.

NH₄—N content in wastewater influent: 100 mg/l or 125 mg/l

Volumetric NH₄—N loading rate: 0.48 kg/m³/day

Mean residence time: 5 to 6 hours

Reactor temperature: 35° C.

Influent pH: 7.5 to 7.7

Air supply rate: 0.06 vvm

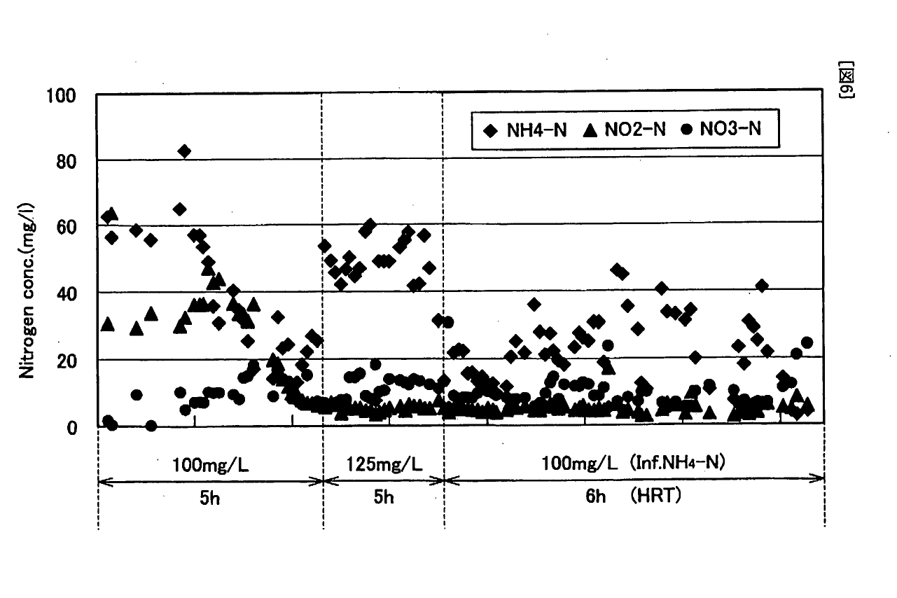

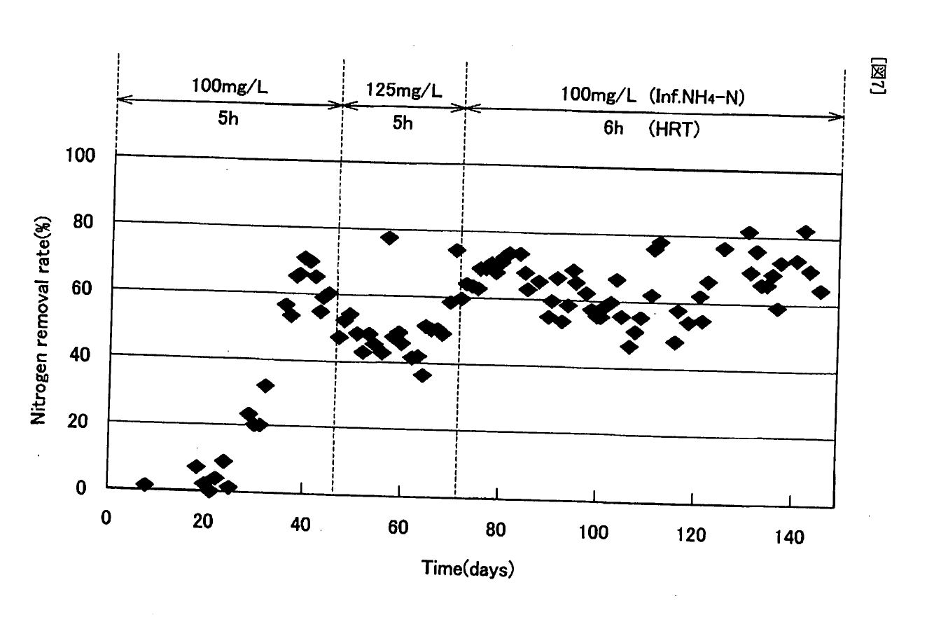

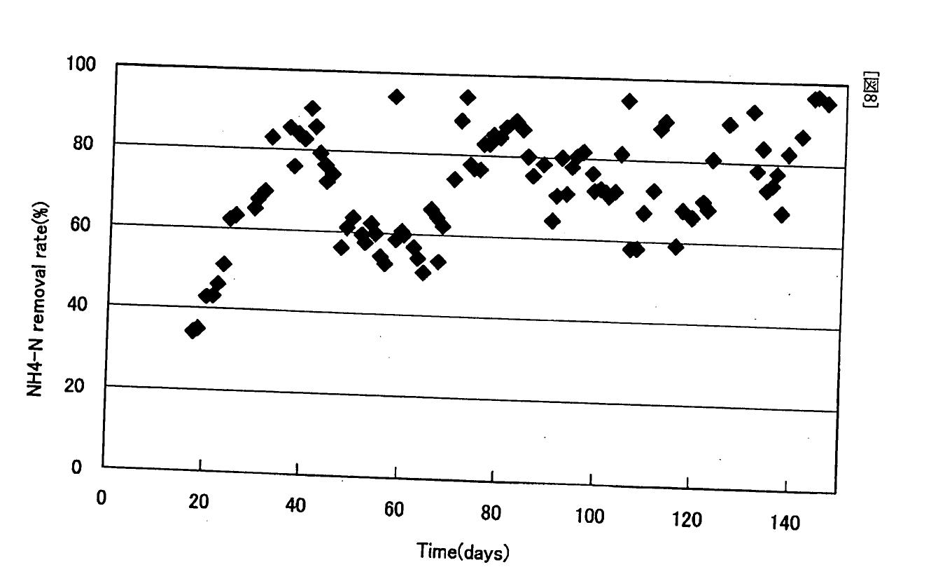

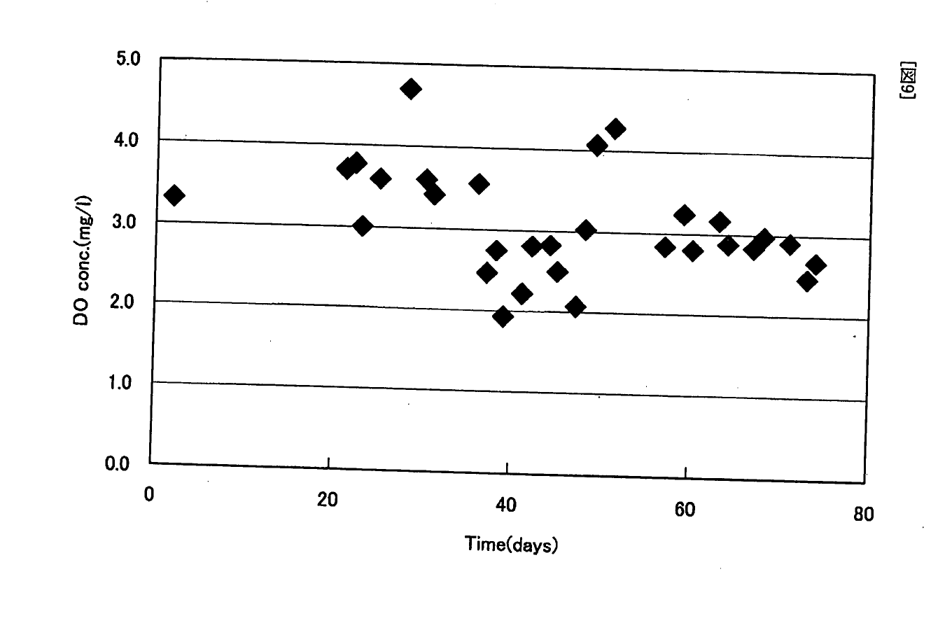

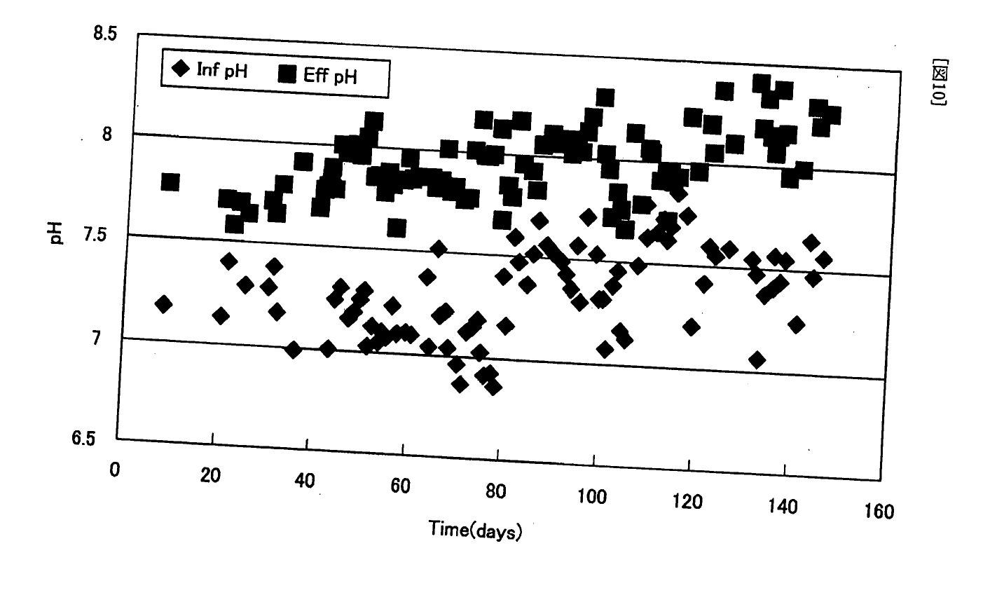

FIG. 6 shows concentrations of NH₄—N, NO₂—N and NO₃—N in treated wastewater. FIG. 7 shows nitrogen removal (%), and FIG. 8 shows NH₄—N removal (%). FIG. 9 shows DO concentrations in wastewater, and FIG. 10 shows pH of wastewater influent and wastewater effluent.

The maximum nitrogen removal was 82%. Immediately after initiation of the continuous treatment, the influent pH was approximately 7.2, and effluent pH was approximately 7.7. After about 50 days from the initiation of the continuous treatment, the effluent pH increased to approximately 8.0 despite pH control in the reaction tank. This indicated that the anammox reaction was in progress and NH₄—N in the wastewater was being removed.

(Photomicrographs of Autotrophic Ammonia-Oxidizing Bacteria and Autotrophic Anammox Bacteria)

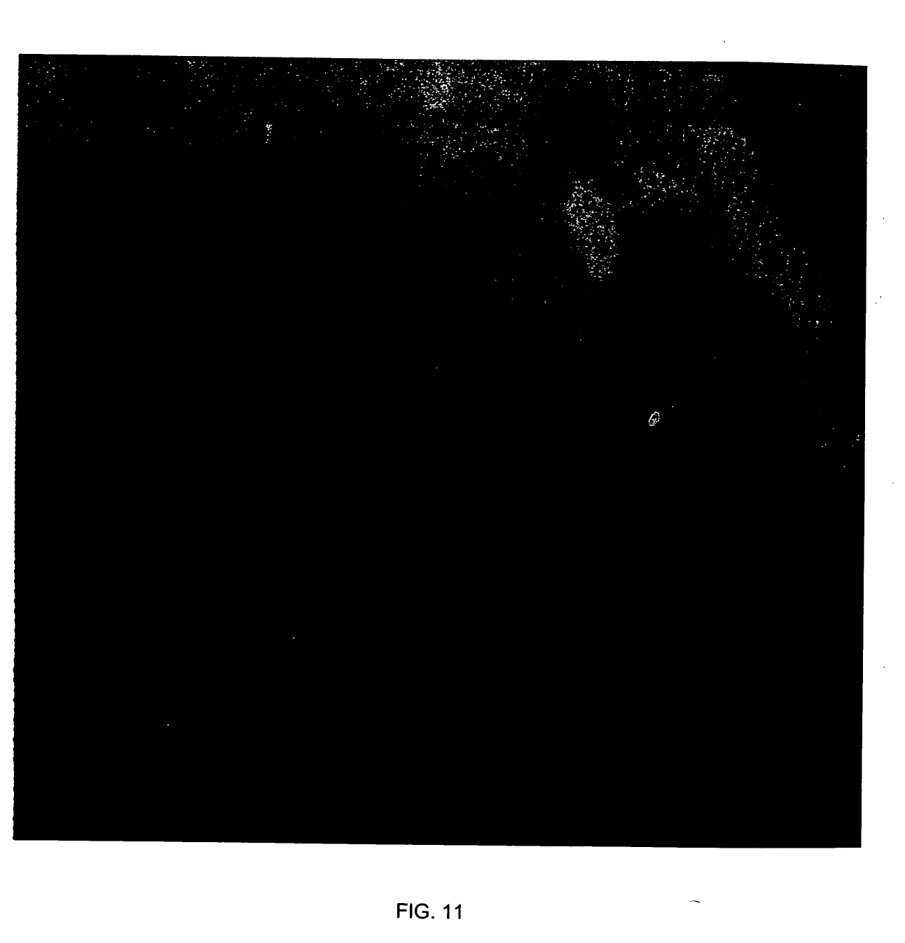

After the above continuous treatment, part of the ammonia-treating material (A) was collected and its biomass was stained by the FISH (fluorescence in situ hybridization) method, and a photomicrograph was taken. The picture is given in FIG. 11. The autotrophic anammox bacteria were stained red, and the autotrophic ammonia-oxidizing bacteria were stained green.

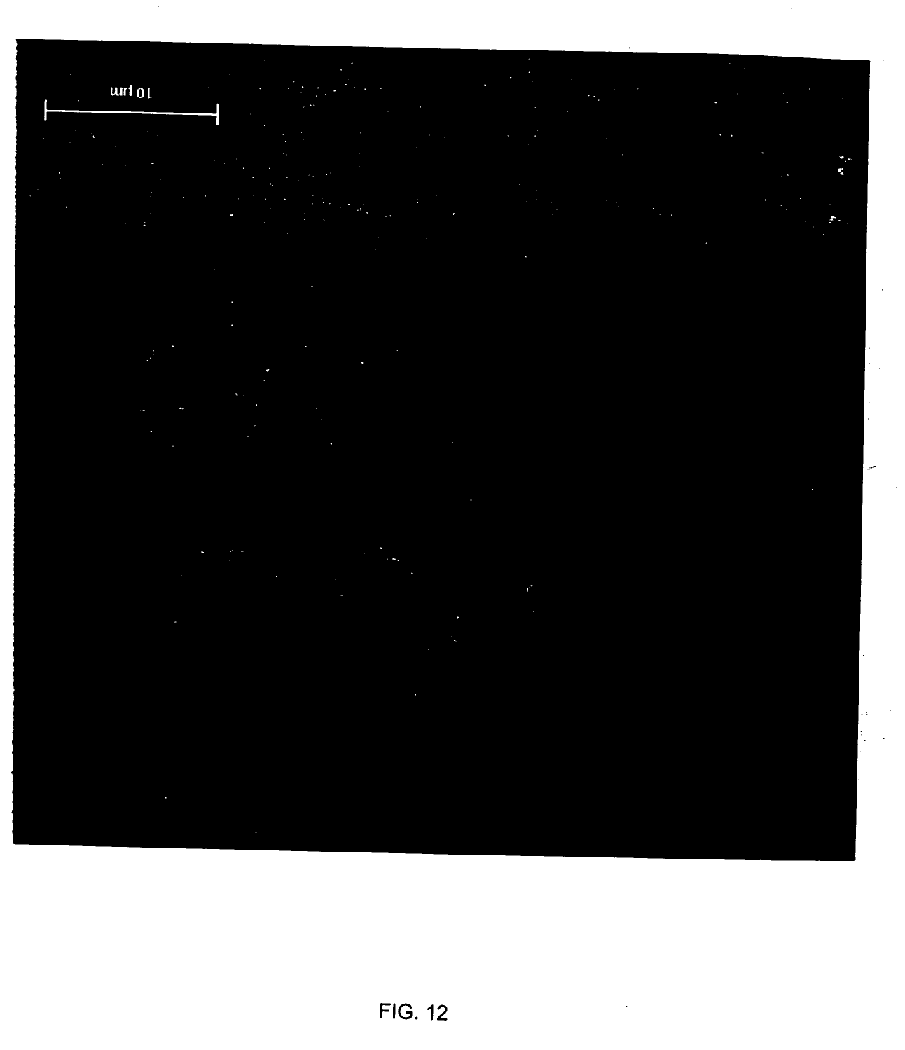

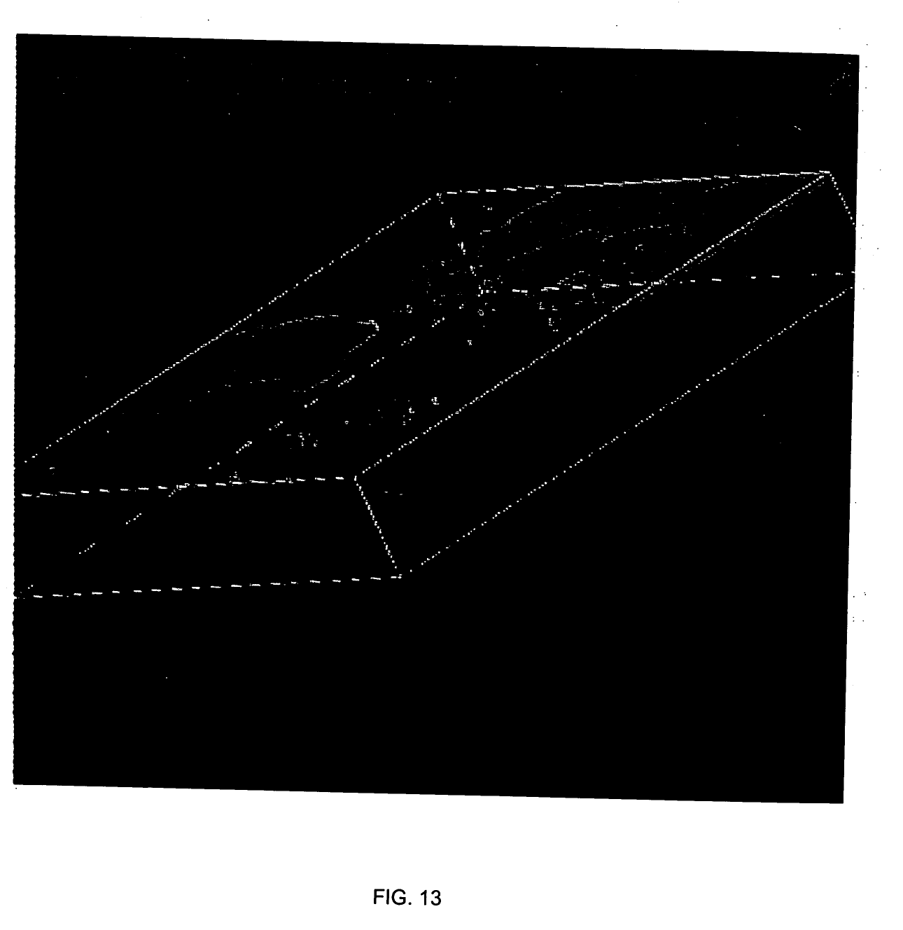

FIGS. 12 and 13 are confocal laser scanning microphotographs of the ammonia-treating material (A).

The FISH photomicrographs and confocal laser scanning microphotographs showed that the autotrophic ammonia-oxidizing bacteria and the autotrophic anammox bacteria were coexisted on the carriers of the ammonia-treating material (A). The autotrophic ammonia-oxidizing bacteria and the autotrophic anammox bacteria existed distinctively from each other, the former being found in an area from 0 to 5 mm from the surface of complex bacterial sludge, and the latter in an area from 5 to 10 mm.

(Identification of Autotrophic Anammox Bacteria)

The bacterial sludge were collected from the ammonia-treating material (A) used in the continuous treatment and were analyzed. DNA of the bacteria collected was amplified by PCR method, and the homology was examined in the internet website of National Center for Biotechnology Information (NCBI), resulting in 100% and 88% homologies with anammox bacteria KSU-1 (AB057453.1) previously found by the present inventors.

### Example 2

Wastewater was treated as described in Example 1 under the following conditions.

NH₄—N content in wastewater influent: 240 mg/l

Volumetric NH₄—N load rate: 0.58 kg/m³/day

Mean residence time: 6 to 10 hours

Reactor DO concentration: 2 to 3 mg/l

Reactor temperature: 32.5 to 35° C.

Influent pH: 7.5 to 8.0

Air supply rate: 0.06 to 0.14 vvm

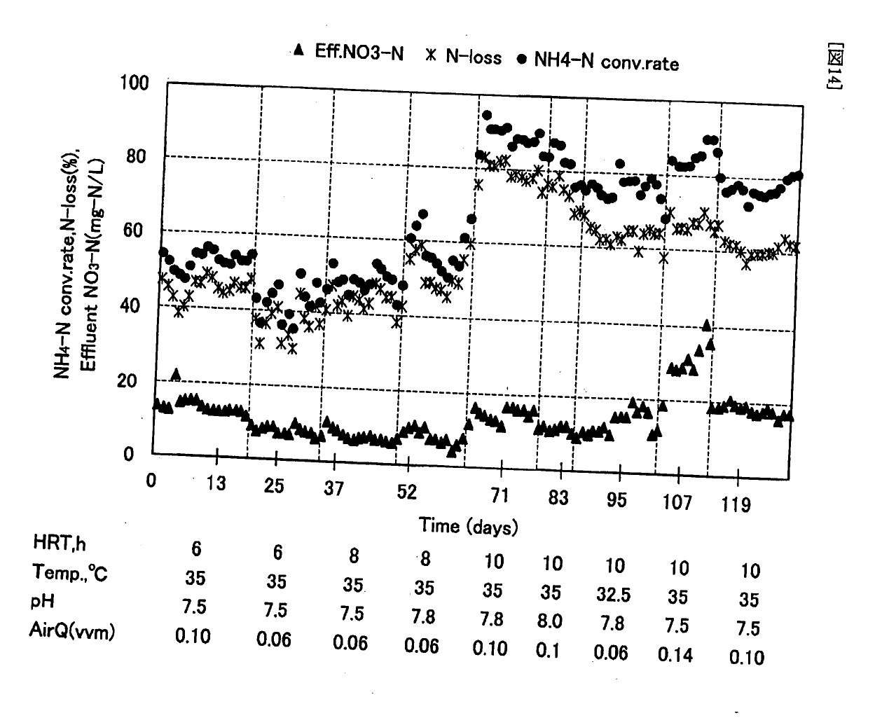

FIG. 14 shows concentrations of NO₃—N in treated wastewater, and nitrogen removal (%).

The maximum nitrogen removal was 80%. The NH₄—N content and DO concentration in wastewater influent were increased as compared to those in Example 1, but the anammox reaction took place and NH₄—N in the wastewater was removed.

### Example 3

Wastewater was treated as described in Example 1 under the following conditions.

NH₄—N content in wastewater influent: 500 mg/l

Volumetric NH₄—N load rate: 1.00 kg/m³/day

Mean residence time: 12 hours

Reactor DO concentration: 2 to 3 mg/l

Reactor temperature: 35° C.

Influent pH: 7.5 to 7.8

Air supply rate: 0.10 vvm

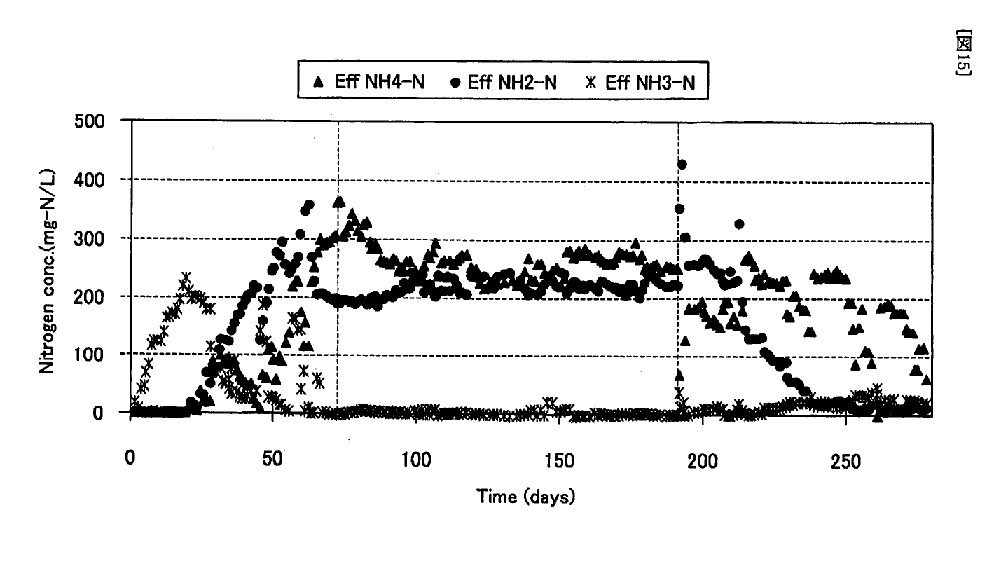

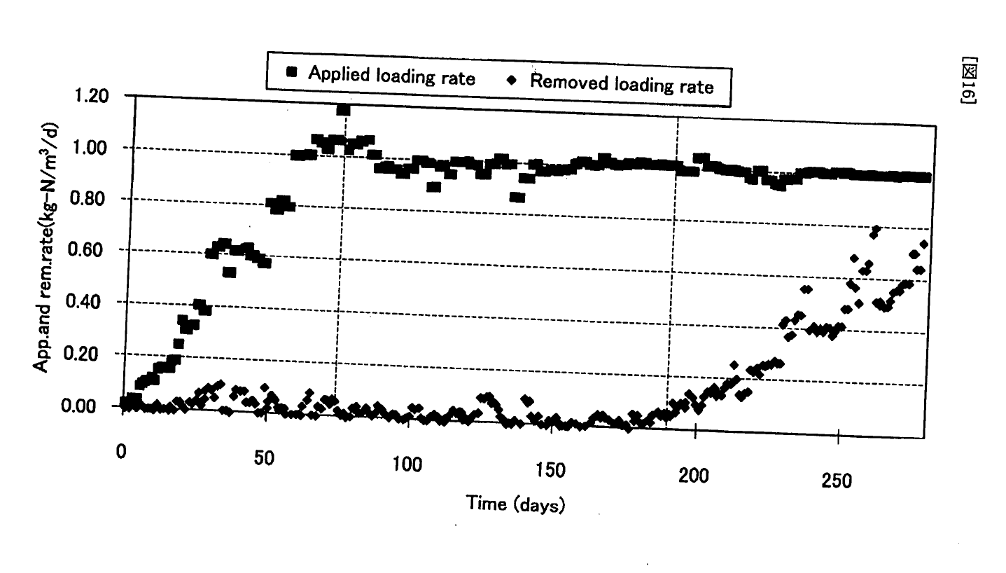

FIG. 15 shows concentrations of NH₄—N, NO₂—N and NO₃—N in treated wastewater. FIG. 16 shows NH₄—N supply and nitrogen removal in treated wastewater.

The maximum nitrogen removal was 80%. The NH₄—N content and DO concentration in wastewater influent were increased as compared to those in Examples 1 and 2, but the anammox reaction took place and NH₄—N in the wastewater was removed.

As described above, wastewater was treated using the ammonia-treating material that included the long carrier net of polyacrylic filaments attached to the supports, and the bacteria attached and immobilized on the net. During the treatment, wastewater flows were produced by aeration. The results of Examples 1 to 3 showed that the wastewater treatment was capable of removing NH₄—N even when the wastewater had a high DO concentration.

### Reference Example 2

### Production Example 2 of Ammonia-Treating Material

(Long Carrier and Reaction Apparatus)

An ammonia-treating material (B) was produced in the same manner as in Production Example 1, except that a reaction tank was used which was 400 mm in height, 260 mm in width, 110 mm in depth and 8 l in reaction part volume.

(Attachment and Immobilization of Autotrophic Ammonia-Oxidizing Bacteria and Autotrophic Anammox Bacteria)

4 g of sludge including autotrophic anammox bacteria was added to 8 L of water, and a mixed liquor suspended solid (MLSS) having a concentration of approximately 500 mg/l was obtained. 20 g of sludge including autotrophic ammonia-oxidizing bacteria was added to 8 L of water, and a mixed liquor suspended solid (MLSS) having a concentration of approximately 2500 mg/l was obtained.

Table 5 shows the composition of influent water medium used in the attachment and immobilization of the sludge including autotrophic anammox bacteria and the sludge including autotrophic ammonia-oxidizing bacteria.

TABLE 5

| Component | Concentration (mg/l) |
|---|---|
| (NH₄)₂SO₄ | 236.0 to 472.0 |
| KH₂PO₄ | 54.4 |
| KHCO₃ | 125.1 |
| FeSO₄•7H₂O | 9.0 |
| EDTA | 5.0 |
| KCl | 1.4 |
| NaCl | 1.0 |
| CaCl₂•2H₂O | 1.4 |
| MgSO₄•7H₂O | 1.0 |

The aqueous solution of the sludge including autotrophic anammox bacteria (MLSS concentration: approximately 500 mg/l) was supplied to the reaction tank. N₂ gas was continuously supplied from a central bottom part of the reaction tank. The pH in the reaction tank was controlled with a pH controller (NPH-690D), and the reactor temperature with a thermostat. The pH was adjusted by automatic addition of a 0.5 mol/l NaHCO₃ solution. The sludge was substantially attached and immobilized on the long carriers in about 6 hours.

The aqueous solution of the sludge including autotrophic ammonia-oxidizing bacteria (MLSS concentration: approximately 2500 mg/l) was supplied to the reaction tank. Air was continuously supplied from a central bottom part of the reaction tank. The sludge was substantially attached and immobilized on the long carriers in about 6 hours.

Consequently, an ammonia-treating material (B) was produced.

### Example 4

Ammonia containing wastewater having a NH₄—N concentration of 50 mg/l was continuously treated for 14 days using the ammonia-treating material (B) produced in Reference Example 2, at a pH of 7.5 and a water temperature in reaction tank of 35° C., and with a mean residence time of 12 hours.

The wastewater was continuously treated for another 66 days, that is, a total of 80 days combined with the first 14 days. The wastewater treatment conditions were as follows. On the 55th day from the initiation of the continuous treatment, an aqueous solution of the sludge including autotrophic anammox bacteria was added with a MLSS concentration of approximately 250 mg/l.

NH₄—N content in wastewater influent: 100 mg/l or 125 mg/l

Volumetric NH₄—N loading rate: 0.5 kg/m³/day

Mean residence time: 6 hours

Reactor DO concentration: 2 to 3 mg/l

Reactor temperature of wastewater in reaction tank: 35° C.

Influent pH: 7.4 to 7.8

Air supply rate: 0.055 vvm

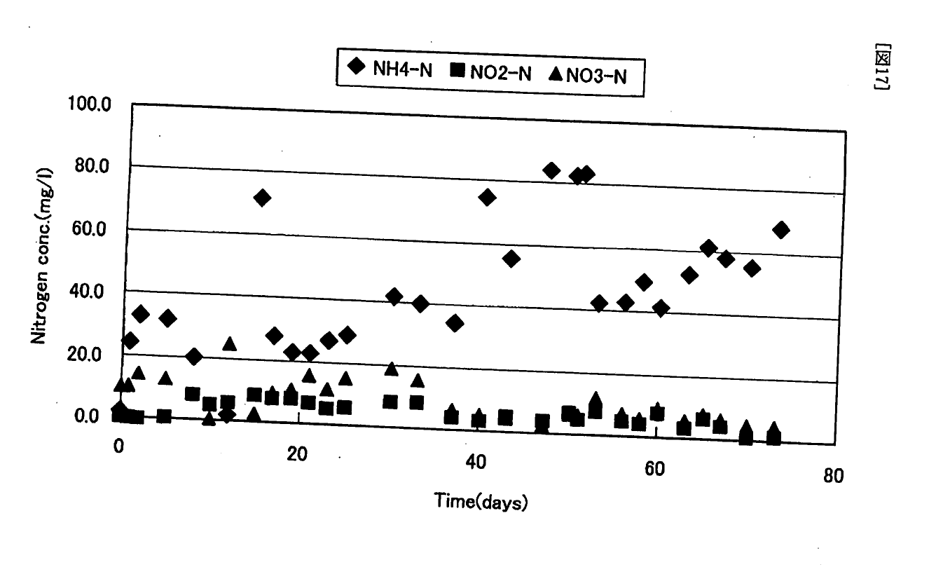

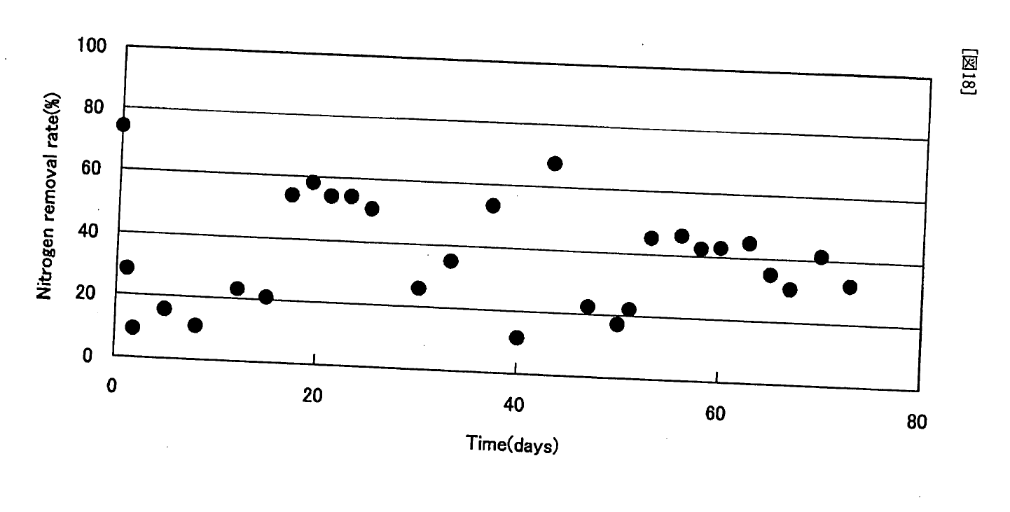

FIG. 17 shows concentrations of NH₄—N, NO₂—N and NO₃—N in treated wastewater. FIG. 18 shows nitrogen removal (%).

The maximum nitrogen removal was 70%. This result indicated that the anammox reaction took place and ammonia in the wastewater was removed.

As described above, wastewater was treated using the ammonia-treating material that included the long carrier net of polyacrylic filaments attached to the supports, and the bacteria attached and immobilized on the net. During the treatment, wastewater flows were produced by aeration. The results of Example 4 showed that the wastewater treatment was capable of removing NH₄—N even when the autotrophic ammonia-oxidizing bacteria and the autotrophic anammox bacteria were attached and immobilized separately.

1. A process for treating ammonia containing wastewater comprising bringing an ammonia-treating material and ammonia containing wastewater into contact with each other to remove ammonia in the wastewater continuously as nitrogen gas,
the ammonia-treating material comprising a long carrier and complex bacterial sludge attached and immobilized on the carrier, the carrier comprising a net, a nonwoven fabric or a woven fabric comprising fibers or filaments, the carrier being attached to a support, the complex bacterial sludge comprising bacteria including autotrophic anammox bacteria and bacteria including autotrophic ammonia-oxidizing bacteria,
the ammonia containing wastewater containing dissolved oxygen at a concentration of not less than 0.5 mg/l.

2. The process for treating ammonia containing wastewater according to claim 1, wherein the bacterial sludge including autotrophic anammox bacteria are attached and immobilized on the fibers or filaments, and the bacterial sludge including autotrophic ammonia-oxidizing bacteria are attached and immobilized on an outer surface of the bacterial sludge including autotrophic anammox bacteria.

3. The process for treating ammonia containing wastewater according to claim 1, wherein in the complex bacterial sludge, the bacterial sludge including autotrophic anammox bacteria are present within the bacterial sludge including autotrophic ammonia-oxidizing bacteria.

4. The process for treating ammonia containing wastewater according to claim 1, wherein the ammonia-treating material and the ammonia containing wastewater are brought into contact with each other in one step.

5. The process for treating ammonia containing wastewater according to claim 1, wherein the ammonia-treating material and the ammonia containing wastewater are brought into contact with each other while supplying air to the ammonia containing wastewater.

6. The process for treating ammonia containing wastewater according to claim 1, wherein the ammonia-treating material is provided in an inner peripheral area in a reaction tank, the ammonia containing wastewater is supplied to the reaction tank, and air is supplied from a central bottom part of the reaction tank to achieve a dissolved oxygen concentration of not less than 0.5 mg/l.

7. The process for treating ammonia containing wastewater according to claim 6, wherein air is supplied from a central bottom part of the reaction tank to generate an upward flow of wastewater in a central area in the reaction tank and a downward flow of wastewater in an inner peripheral area in the reaction tank.

8. The process for treating ammonia containing wastewater according to claim 7, wherein an air guide tube is provided in a central area in the reaction tank in a position such that a lower opening of the tube is opposed to the bottom of the reaction tank with a space from the bottom of the reaction tank, and air is supplied through the lower opening of the air guide tube to generate an upward flow of wastewater in the central area in the reaction tank.

9. The process for treating ammonia containing wastewater according to claim 6, wherein the longer direction of the long carrier is perpendicular to the bottom of the reaction tank.

10. The process for treating ammonia containing wastewater according to claim 1, wherein the fibers or the filaments are polyacrylic fibers or polyacrylic filaments.

11. The process for treating ammonia containing wastewater according to claim 1, wherein the long carrier has a length to diameter ratio of not less than 3.

12. The process for treating ammonia containing wastewater according to claim 1, wherein the bacterial sludge including autotrophic ammonia-oxidizing bacteria are attached and immobilized in a thickness of not less than 5 mm.

13. The process for treating ammonia containing wastewater according to claim 1, wherein the ammonia containing wastewater in the reaction tank has a BOD concentration of not more than 20 mg/l.

14. The process for treating ammonia containing wastewater according to claim 1, wherein the ammonia containing wastewater in the reaction tank has a temperature of 30 to 40° C.

15. The process for treating ammonia containing wastewater according to claim 1, wherein, the ammonia containing wastewater in the reaction tank has a pH of 7.4 to 8.0.
